(19) United States
(12) Patent Application Publication
(10) Pub. No.: US 2004/0132008 A1
(43) Pub. Date: Jul. 8, 2004

(54) Elucidation of gene function

(76) Inventor: Benjamin J Doranz, Philadelphia, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/476,297

(22) PCT Filed: Apr. 30, 2002
(86) PCT No.: PCT/US02/13432

(51) Int. Cl. C12Q 1/70; C12Q 1/68; G01N 33/48

(52) U.S. Cl. 435/5; 435/6; 435/40.5

(57) ABSTRACT

Articles and methods are provided for determining the function of genes in a rapid and cost effective manner. Nucleic acids are arrayed upon a substrate. In accordance with certain preferred embodiments, viable cells are subsequently caused to be bound to the substrate at the locations occupied by the nucleic acids. Subsequent transduction or transfection of the cells by the nucleic acids followed by continued vitality of the cells permits expression of the proteins encoded by the respected nucleic acids. Knowledge of the identity of the nucleic acids, at least as regards their locations on the substrate, permits determination of protein function thereof. Methods of creating and using such cell-arrays, and methods of reverse-transfection and reverse-transduction are featured.

### BACKGROUND OF THE INVENTION

Genes are the blueprints of all living organisms and are physically composed of DNA. The collection of all genes of an organism is called a genome. When “expressed,” each gene is translated into a distinct protein, and proteins are the physical building blocks of all living organisms. Each cell in an organism is composed of tens of thousands of proteins, each of which has a function that, collectively, defines what that cell does and how it behaves.

Gene expression identifies which genes are used in any given cell type, and how often each of those genes is used. When genes are active, that is, “expressed,” they make copies of themselves, called messenger RNAs (mRNAs), which in turn direct the production of their protein products. Gene expression technology identifies and quantifies all of the mRNAs in a cell. Different cell types use different subsets of genes. It is the subset of genes and how often each gene within the subset is used that defines cell function, that constitutes its “biological program.”

Each of the 30,000-60,000 genes that each human carries (the human “genome”) encodes for a distinct biological function. These functions are carried out not by the genes themselves, but by their protein products (the human “proteome”). A gene encodes for the production of a protein, and that protein performs that gene's biological function. Several factors make it necessary to study proteins, rather than just genes themselves, in order to arrive at a complete understanding of normal biology and disease mechanisms. For example, drugs act on proteins, not genes, so an understanding of protein structure and function is crucial to rational drug design and optimization. In addition, the correlation between mRNA levels and the abundance of the encoded protein is very poor. Thus, while genomic data can provide clues to functional differences between two biological states, the measurement of differences at the protein level reveals true discoveries.

Proteomics is a term that describes the study of proteins—their expression, interactions, and structure/function relationships—within the context of the framework provided by genomics. Whereas genomics is devoted to identifying all human genes, proteomics will be crucial to the development of the higher order information necessary to understand how these genes function.

Until recently, researchers focused on the identification and sequencing of genes involved in a specific disease. However, the mere identification of a disease-related gene is not sufficient to understand its role in the disease process. Information on the role of an individual gene or a set of genes in the complex biology of a particular pathological process is essential. This is where functional genomics will play a key role.

Functional genomics aims at assigning the function to genes that are responsible for specific biological processes and diseases. It meets the challenge to identity new and clinically relevant drug targets and therapeutic genes. Currently, the industry is limited in their drug development efforts by the lack of new validated drug targets. Of the genes identified to date, the function of only a small number has been determine Various techniques have been employed thus far to assign function to a gene. However, ascertaining the direct link between genes and their biological function remains a major technical hurdle. The pharmaceutical and biotechnology industry is looking for a fast, efficient and automated technology platform to identify those genes that have diagnostic, therapeutic research, and other relevance.

The challenge pharmaceutical companies face today is to develop drugs that act on novel, specific protein targets that are produced by genes. Despite revolutionary advances made in molecular biology and genomics, until recently only approximately 400 out of about 4,000 possible targets have been identified. Moreover, there has been no fast and efficient way to identify additional targets for drug development. Efforts to sequence the complete set of human genes have generated huge amounts of fundamentally important genetic information, including useful information about a handful of genes that are associated with particular disease conditions. However, there has been limited progress using this information to identify drug targets quickly and systematically. The result is a shortage of validated drug targets and a dearth of tools to determine which new targets have clinical promise.

One problem is that a human cell is vastly more complex than a linear arrangement of genes that systematically “pump out” their proteins. Only a small subset of possible proteins is made in a given cell at a given time and this subset changes over time and with environmental conditions. With 30,000 genes, the number of possible combinations of expressed proteins is staggering, and often the answer may lie in their interaction or regulation, not just in their expression. Many questions related to finding drugs have remained. How do the different proteins produced by these genes interact in various parts of the cell to result in a particular biological outcome, like releasing histamine in allergic individuals, or multiplying without retaining the characteristics of the cell's organ, such as a lung cell multiplying into tumor cells? How does the folded, three-dimensional shape of a protein (beyond its linear, two-dimensional sequence) effect the biology of the cell? Most importantly, what is the function of all these genes in relevant disease processes? An understanding of the key biological “relationships” in the disease process is still missing but is much needed: when, to what degree, and under what conditions (i.e., in what disease states) are various combinations of genes expressed and what are the key relationships among these genes? Many other questions of this kind also are extant Tools and methods for addressing such questions are greatly desired.

Genomics in species other than man is developing as well and the need thus exists for ways to ascertain gene function in such systems. For example, knowledge of gene function and relationship in insect species will provide improved, selective, pesticides and the like. Understanding animal gene function will permit development of industrial, commercial, consumer and other products and methods having a decreased environmental burden than at present, while obtaining improved efficacy and efficiency. Veterinary products and other materials will be improved thereby. Nor are the benefits of knowledge of gene function limited to animal systems. The genes of plants, fungi, viruses, bacteria and even prion-like constructs may be elucidated in this way. Great economic, therapeutic and environmental benefits result.

Although these approaches can tell us what genes or gene products are “involved” in a disease state (i.e. they were expressed in some pattern statistically related to that phenotype), they could not tell us which, if any, caused the condition—or—whether the converse was instead true. Also, because of the complex nature of the interactions of molecules within the cell, even if a gene that was present in a disease state could be identified, redundancies in the biology or slight mutations in a gene provided for almost unlimited permutations and combinations of outcomes. Moreover, researchers still do not know what will reverse the disease condition, the real goal of drug therapy.

Once any of these approaches has produced some information about the genes that are “involved” in a disease state, they all share a time—and resource—consuming next step. Since involvement is not causality, researchers do not know which gene or gene product causes the disease, much less which can cause its reversal. Within the context of drug discovery, this like is termed “target validation.”

Realizing that the answer to finding new drug targets that can reverse the effects of disease may lie in the interaction between proteins, not just the over- or under-expression of them, researchers have begun to study the function of different genes and proteins and how those proteins function within the context of the entire signaling pathway to which they belong. As part of this effort, scientists are also beginning to elucidate the signaling pathways (intricate biochemical circuits of proteins that relay massages to the cell) to determine how that interplay—affects biological outcome of the cell. This attempt to ascertain the biological function of genes and their protein products is known as “functional genomics.” Many functional genomics approaches involve conducting assays (laboratory tests) to determine the function each protein in a pathway of interest, then moving onto the next pathway and analyzing its members, and searching through the complicated myriad of pathways, by process of elimination, for a protein target that regulates function.

Given the seemingly endless number of proteins that could be involved with a particular disease, this approach is incredibly time-intensive and inefficient In addition, it frequently leads to a dead end for two primary reasons. First, often it is not an individual protein that controls the biological fate of a cell, but its interaction with another protein that is the key event. Further complicating matters is that in each cell, there exist many possible signaling pathways that can lead to a variety of physiological outcomes. In treating disease, it may be possible to modify a signaling pathway other than the defective one and still improve the health of the cell. Also, the proteins and pathways selected for these studies are based on an assumption that they are “involved” in a disease and not any true biological scientific evidence that they are causally related. Given the cost (over $500 million per drug) of the subsequent steps from small molecule screening through animal testing to human trials and the time used (6-12 years), this can be an expensive and time-consuming gamble. Since these failures are usually because of toxicity or lack of efficacy (functional reflections of the target's activities), functional information at the very beginning of the discovery process could avoid much of this wasted time and money.

There are two general approaches to associating protein function with gene structure. The first approach involves deciphering a particular gene sequence from a vast amount of genetic data, cloning the gene, modifying the cloned gene so that it actively expresses the protein it encodes and then screening this protein for biological function. The second approach involves the initial identification of a tissue or cell type that exhibits a biological characteristic of interest, isolation and identification of the proteins involved, and then identification of the genes that encode such proteins. Major limitations of both methodologies are that they are typically resource-intensive, involve multiple time-consuming steps, and generally require the identification and cloning of the gene or knowledge of a gene's sequence in order to produce protein. Because the protein is the functional unit of life, the production of protein for functional analysis is one of the most significant bottlenecks in the development of new gene-based therapeutic and diagnostic products.

Thus, there exists a great need for ways to ascertain the function and relationships of genes in a rapid and economical way. Such methods and the associated tools, protocols and materials are greatly sought in order to provide commercially valuable information for drug discovery, diagnostics, veterinary products, pesticides, fungicides, industrial materials, commercial materials, consumer products and otherwise.

### SUMMARY OF THE INVENTION

The present invention is directed to achieving some or all of the foregoing objectives as well as other objects and benefits as will be apparent to persons skilled in the art. In an exemplary embodiment, arrays of viable cells are provided on a substrate. The array comprises elements, each of the elements comprising a subset of the cells. The cell are transfected or transduced with nucleic acid, preferably a preselected nucleic acid. The identity of the nucleic acid is known, at least in relation to the location of the array element with which it is associated on the substrate. The nucleic acids may be DNA or RNA, may be encapsulated within a gene delivery vector such as a virus, and may—and in accordance with certain preferred embodiments do—comprise all or part of a library.

Such libraries may comprise cDNA libraries, RNA libraries, oligonucleotide libraries, antisense libraries, viral libraries, and other libraries and library-like collections. In accordance with other embodiments, the molecular identity of at least some of the nucleic acids is known. In accordance with other embodiments, the nucleic acids are selected for their likelihood of inhibiting, stimulating or otherwise affecting a disease state, phenotype or condition. Such may be selected for association with known or suspected biological functions as well. The nucleic acids may be frozen prior to their having been transfected into the viable cells and, indeed, the long-term stability of arrays of such nucleic acids on substrates permit efficient and convenient elaboration of viable, transduced cell-arrays upon demand.

The nucleic acids may be mammalian, especially human, and can represent a wide range of species including rabbit, rodent, primate and others. Non-mammalian animals such as insects, fish, birds, and lower creatures may also give rise to the nucleic acids. Such may also derive from plants, fungi, bacteria, viruses and even constructs such as prions and the like. They may be wholly-artificial as well and may include any of a wide variety of homologies, substitutions, variants, and chemically modified species—all known, per se, to persons skilled in the art.

It is preferred that a gene transduction vehicle or promoter be provided attendant to the nucleic acids to facilitate transduction into and expression within viable cells with high efficiency.

This invention is also directed to arrays of nucleic acids on a substrate, the identity of each of the elements of the array being known at least in relation to the location of each element on the substrate. Such arrays include gene transduction vehicles or promoter attendant to the nucleic acids. It is also preferred for some embodiments that binding of nucleic acids to the substrate is enhanced through provision of a binding enhancement material on the substrate or with the nucleic acids themselves.

These arrays of nucleic acids are prepared by elaborating upon a suitable substrate nucleic acids in a preselected pattern or spatial arrangement. The nucleic acids are preferably associated with a transduction promotion vehicle or agent These arrays can be stored for long periods of time, especially when frozen. The arrays of nucleic acids on a substrate may be further manipulated—even after passage of time—by binding viable cells to the substrate at the locations where elements of the array of nucleic acids are present The cells are caused to become transduced by the nucleic acids such that an array of viable cells having exogenous nucleic acid therein arises.

It will be understood that the terms “transfection” and “transduction” are closely related in the art and that the former term is generally “contained within” the latter. They will be used generally as synonyms, but otherwise, as convention in the scientific context suggests.

These arrays of cells can be used in a very wide number of assays, screens and tests, especially including protocols which elucidate the function of the genes represented by the genetic material thus provided. Thus, in a preferred embodiment, arrays of cells on a substrate can be incubated under conditions selected to promote their growth in order to give rise to the biological products coded for by the nucleic acid incorporated into the cells. Determination of these products can be correlated with the identity of the nucleic acid by virtue of the spatial position of the respective cells on the substrate, such that the functions of the nucleic acids can be elucidated and attributed to the particular nucleic acid involved.

A very wide range of screens, tests and assays may be used with the arrays of this invention. It is preferred that such activities be conducted under the operative control of a computer.

The invention includes an array of viable cells on a substrate, each element of the array comprising a subset of the cells transduced with a preselected nucleic acid, and the identity of each of the transduced nucleic acids being known in relation to the location of the element on the substrate. In one aspect, the preselected nucleic acids comprise a cDNA library, a viral vector library, an RNA library, an oligonucleotide library, a library from a virus, an agrobacterium library, or an antisense library. In another aspect, the preselected nucleic acids comprise the identical gene mutated at defined genetic locations at each element of the array. In another aspect, the identities of at least some of the preselected nucleic acids are known In another aspect, the preselected nucleic acids have been frozen on the substrate. In another aspect, the preselected nucleic acids are selected for their likelihood of inhibiting an identified disease state, phenotype or condition. In another aspect, the preselected nucleic acids are selected for their being associated with a known or suspected biological function. In another aspect, the preselected nucleic acids encode membrane proteins. In a further aspect, the preselected nucleic acids encode G-protein couple receptors, ion channels, or viral Envelope proteins. In another aspect, the cells are mammalian, avian, insect, plant, plant protoplast, yeast, fungus, bacterium, or human. In another aspect, the cells stably express a gene of known identity prior to their application to the substrate.

The invention also includes an array of nucleic acids on a substrate, the identity of the elements of the array being known in relation to the location of the elements on the substrate, and each element of the array further comprising a gene transduction vehicle. In one aspect, the nucleic acids are DNA. In another aspect, the nucleic acids comprise a cDNA library, a viral vector library, an RNA library, an oligonucleotide library, a library from a virus, an agrobacterium library, or an antisense library. In another aspect, the nucleic acids comprise the identical gene mutated at defined genetic locations at each element of the array. In another aspect, the identities of at least some of the nucleic acids are known. In another aspect, the array is substantially stable to freezing conditions. In another aspect, the substrate is compatible with use for mass spectrometry. In a further aspect, the substrate further comprises a gold layer.

The invention also includes a solid body having a surface, the surface being adapted to bind a gene transduction vehicle in a reversible manner, to permit cells to adhere to the surface, and to allow cells to be transduced by the transduction vehicle. In one aspect, the adaptation comprises an antibody directed to the transduction vehicle. In another aspect, the adaptation comprises an antibody directed to the exterior proteins of a viral vector. In a further aspect, the adaptation comprises an antibody directed to the Hexon or Fiber proteins of Adenovirus.

The invention also includes a solid body having a surface treated to allow spotting of volumes of liquid less than about 1 microliter in an array format without allowing the liquid to completely desiccate. In an exemplary aspect, the treatment comprises application of trehalose or gama-amino-propylsilane, or freezing of the array during or after spotting of liquids.

The invention also includes a method for spotting volumes of liquid less than about 1 microliter in an array format onto a solid surface without allowing the liquid to completely desiccate, comprising including in the spotting medium a sugar, trehalose or glycerol.

The invention also includes a method of constructing an array of viable cells. The method comprises providing a substrate, elaborating upon the substrate an array of nucleic acids, binding viable cells to the substrate at the locations where elements of the array of nucleic acids are present, and transducing at least some of the cells present at said locations with the nucleic acid present at those locations. In one aspect, a gene transduction enhancing composition is included with the nucleic acids elaborated upon the substrate. In another aspect, a surface of the substrate is coated with a binding promoting composition to enhance the binding of the array of nucleic acids to the substrate. In another aspect, the array is incubated subsequent to transduction under conditions selected to promote growth of the cells. In another aspect, the cells are mammalian, rodent, rabbit, primate, avian, insect, plant, plant protoplast, yeast, fungus, bacterium, or human. In another aspect, the substrate is inorganic or glass material.

The invention also includes a method for determining the biological products produced by members of a library of nucleic acids. The method comprises constructing an array of viable cells by elaborating upon a substrate an array comprising at least a portion of said library of nucleic acids, binding viable cells to the substrate at the locations where elements of the array of nucleic acids are present, transducing at least some of the cells present at said locations with the nucleic acid present at those locations, incubating the array of cells under conditions selected to promote growth of the cells, determining the biological products produced at elements of the array, and relating the production of such elements with the nucleic acid present at said elements. In one aspect, the identities of the members of the library of nucleic acids are known in relation to the location of the nucleic acids on the substrate. In another aspect, the library of nucleic acids is selected to be related to a disease state. In another aspect, the library of nucleic acids is selected to be associated with a known or suspected biological function. In another aspect, the library of nucleic acids is used to identify a protein mutation with a defined phenotype or function.

In another aspect, the library of nucleic acids is selected to encode surface-bound monoclonal antibodies. According to one preferred embodiment, the array is used to identify drug candidates that bind to proteins correlated with adverse absorption, digestion, metabolism, excretion, toxicity, bioavailability, or cell death. In another aspect, the library of nucleic acids comprises the identical gene mutated at defined genetic locations at each element of the array. In another aspect, elaboration includes placing upon a surface of the substrate a binding promoting composition to enhance the binding of the nucleic acids to the substrate. In another aspect, elaboration includes co-depositing a gene transduction enhancing composition with the nucleic acids on the substrate. In another aspect, relating comprises detecting the biological products produced by the cells. The array can be challenged with a predetermined chemical or biological species during at least part of the incubation step. In another aspect, the array is used to identify a protein mutation with a select phenotype. In a further embodiment, the phenotype is improved antibody reactivity for use in designing improved vaccine candidates. In another aspect, the array is used to identity the target for a drug candidate where said target is not yet linked to a specific disease. Arrays in accordance with the invention can be used to identify the target for a drug candidate of unknown specificity. In a further aspect, the drug candidate is a protein, monoclonal antibody, or low-molecular weight organic compound. In a further aspect, the drug candidate has been tested for toxicity and bioavailability prior to the identification of its target In another aspect, the array is used to define antibody reactivities from an animal's sera. In another aspect, the nucleic acid library is derived from one species and the cells are derived from a different species. In another aspect, the cells stably express a gene of known identity prior to their application to the substrate.

In another aspect, the cells used contain a co-transduced gene, comprising one or more genes introduced into all the cells used on the array. In a further aspect, the co-transduced gene is a modifying enzyme. In a further aspect, the co-transduced gene is a kinase, phosphatase, glycosidase, protease, or chaperone protein. In a further aspect, the co-transduced gene is identified using the methods described above to identify the function of a gene or protein. In such iterative usage, the array is first used to identify a gene that confers a particular phenotype of function upon a cell, that gene is then introduced either stably or transiently into all or substantially all the cells that are then placed on another cell-array to identify a different gene that confers a phenotype or function to the cell that is related in some way to the first gene, either by homology, function, phenotype, complementarity, inhibition, or relatedness on a pathway. The identification of proteins that are involved in the same pathway is one example of such iterative usage.

### DETAILED DESCRIPTION OF CERTAIN PREFERRED EMBODIMENTS

The present invention has been named by the inventor the “Protein Expression Chip” or the “cell-array.” This exemplary cell-array is a preferably disposable array comprising a capture media bonded to a solid surface and an arrayed library of gene transduction vectors. The transduction vectors comprise nucleic acid together with the optional but greatly preferred transduction promotion material. Living cells are placed on the chip overnight, the cells are allowed to express, and any one of hundreds of different assays can then be performed An exemplary cell-array can be constructed as follows:

a. Slides are preferably coated with a substrate to which mammalian cells can bind.

b. The slides are also arrayed with a cDNA library within plasmids or viral vectors. The library could also be in anti-sense form to inhibit natively expressed genes in the cell.

c. The cDNA library is mixed with a gene transduction vehicle that will allow the DNA vector to adhere to the slide but also to transduce the cell.

d. Cells in suspension are plated directly onto this array. Cells that settle onto the slide and adhere over a particular spot (position) in the array will contact the genetic clone in the transduction vector.

e. Using one of several possible molecular mechanisms, cells contacting a specific clone will be transduced. Convenient molecular mechanisms include use of retroviruses, Adenovinises, Vaccinia virus, Adeno-associated Virus, Baculovirus, Semliki Forest Virus, and other viruses which can mediate gene transduction. In addition, chemical and lipid-based transfection methods such as calcium phosphate, DEAE Dextran, transferrin, Lipofectamine, or GenePorter can be used. Any gene transduction vector is possible that can allow the gene to enter the cell and be expressed.

f. The cells, now adhered to the slide surface and transduced with a gene, are allowed to express the gene (e.g. growth at 37° C. overnight).

g. The living cells, now over-expressing a defined functional protein, can then be assayed using any number of techniques. With the correct vector, very high levels of expression can be achieved for biochemical and functional detection.

Once transduction has occurred and the cells have taken in the nucleic acid, e.g. cloned cDNA, the effect of the expression of the cDNA can be observed. The library can be such that the nucleic acids introduced to the cultured cells will have a mechanism to positively effect expression (i.e. the vectors will have a gene promoter). In this manner, the information residing in the cDNA sequence will be expressed Any and all detection methods can then be utilized. Mechanical, optical, and laser array reading possibilities that currently exist are capable of detecting the different signals of output assays from slide-based arrays. When a spot with a desired property (i.e. signal) is detected, its position in the array makes identification of the gene that caused the signal straightforward.

The surfaces that can be used for gene expression and array creation can be composed of any number of solid or semi-solid surfaces that can support the creation of an array and/or the growth of cells. For example, slides can be coated with a substrate to which mammalian cells can bind. Some slide materials do not need to be coated, while others may be coated to increase cell adherence. The surface must also support the creation of an array which can be temporarily bonded to the surface until cells are added and gene transduction occurs. The surface substrate that is used for this reversible DNA adherence may be the same or a different chemical composition than the substrate used to promote cell adherence. The surface may also be created to allow alteration of assay and detection (e.g. conductive material to control hybridization stringency). For example, the electromagnetic properties of some ceramics and metals can be tuned to enhance gene transduction, detection, optical reflectance or transmission, hybridization, or other uses of the array. The array has been enabled using glass, tissue culture plastic, Poly-Lysine coated glass and plastic, and permanox plastic. Other examples of surface materials that can be used include, glass, quartz, ceramic, plastic (e.g. polystyrene, polypropylene), permanox, poly-lysine coated surface materials, silanized surfaces, tissue culture plastic (e.g. polystyrene), agar, dextran, nylon, paper, nitrocellulose, silicon, gold, and optical fiber.

The genetic constructs that introduce DNA into cells for expression can be such that the DNA introduced to the cultured cells will have a mechanism to positively effect expression. In other words, the vectors will preferably have a gene promoter in order to attain efficient expression of protein from that gene. The promoter can be any number of widely used constitutively active promoters, such as CMV, but can also be composed of inducible promoters, cell-type specific promoters, or any other type of transcriptional element In this manner, the information residing in the cDNA sequence will be expressed. The genetic library can be composed of cDNA but could also be composed of a genomic library. The use of cDNA focuses the screen on expressed sequences and is thus superior to random genomic sequences which may or may not be expressed and may or may not be expressed in any given cell type. The library could also be in anti-sense form to inhibit natively expressed genes in the cell. The library could also encode peptide sequences to screen for active or inhibitory functions of peptides, or to measure their ability to bind molecules (e.g. antibodies, T-cell receptors).

For the construction of arrayed cDNA libraries, total mRNA is converted into cDNA, cloned into a transfer vector and subsequently transformed in E. coli. A normalization process (e.g. multiplex hybridization with oligonucleotides) can remove the majority of abundantly expressed genes, resulting in a normalized library. Individual colonies of library are arrayed in a microtiter format (e.g. 96-well). Automated plasmid preps can then amplify the uniform DNA construct within each pick The purified DNA can then be arrayed onto a cell-array. The use of a library from a source of interest (e.g. a tumor, a unique cell line, or cells of phenotypic interest) can be used to identify the genetic and proteomic determinants of cell behavior and phenotype.

The construct used can code for any number of types of proteins, peptides, or gene products, including cDNA, mRNA, ribozymes, RNA-protein fusions, organic compounds, cofactors, secreted proteins, membrane receptors, and others.

The transduction vehicle can be composed of a number of different forms. Any gene transduction vehicle is possible that can allow the gene to enter the cell and be expressed. The critical property of the library is that it exist in the form of a transduction vehicle that will upon the addition of cells, effect the introduction of the individual clones that make up the library, into the cells in the immediate vicinity of the spot (position in the array). Optimal vectors will be determined through experimentation. Alternative vehicles may be required for cells of different species or plant organisms. Several possible molecular mechanisms of gene transduction are possible, including viral vectors, chemical vehicles, bacterial vectors such as agrobacterium, and lipid-based vehicles such as, retroviruses, adenoviruses, vaccinia virus, adeno-associated virus, adenovirus-AAV combination viruses, murine, leukemia virus, HIV and SIV-based vectors, VSV (with or without a low pH-buffer pulse), bacculovirus, semliki forest virus, other viruses can mediate this gene transduction, transposons, adenovirus DNA conjugates, peptide-MAb conjugates, calcium phosphate, DEAE Dextran, transferrin, lipofectin, lipofectamine, lipofectamine plus, lipofectamine 2000, Gene Porter™, PEG, phosphatidylserine and calcium, microinjection, magnetic beads, and ballistic particles (gene guns).

Attachment of a gene transduction vector to the substrate surface can be accomplished using any number of methods that have the effect of maintaining the vector in the approximate location where initially applied while still allowing the vector to enter cells once cells are added. The surface of cells is naturally negatively charged. Purified DNA is also naturally negatively charged. A positively charged intermediate is often used to mediate introduction of purified DNA across a cell membrane and into a cell for expression (i.e. most transfection reagents function in this manner). The chemistry used to attach the gene tansduction vector may also take advantage of these properties, although it is not a necessary feature. One approach is to dry small spots of transfection mixture on a slide surface. Other mechanisms for attachment include, drying, salt precipitate, crystallization capture, biotin/avidin, polyethylene glycol (PEG), polyethylene oxide, biotinylated lipid-doped transfection reagent, tetrameric avidin for binding multiple molecules at once, poly-lysine, glycerol, trehalose, gama-aminopropylsilane, polyethyleneimine, DEAE-dextran, gelatin, pluronics, gum arabic, sucrose, antibody capture, carboxylated polyvinylidene fluoride, dextran and carboxy-dextran, lectins and carbohydrates, cross-linking, covalent modification for attachment (e.g. free amines to carboxy-dextran), electrostatic (charge-mediated) attachment, physical barriers (e.g. etched wells), antibody-mediated attachment, and magnetic attachment For adherence and stability of viral vectors to a surface, preferred embodiments include adherence using antibodies, positively charged compounds, coated surfaces such as GAPS-coated glass, and the addition of stabilizing reagents such as trehalose, gelatin, glycerol, or sucrose. For example, an antibody specific for the Fiber protein that composes the exterior of Adenovirus can be used to capture the virus to a surface but still allow cells to be infected by the vector. Similarly, an antibody specific for the Hexon protein of Adenovirus can accomplish the same results.

DNA adheres to a number of surfaces (glass, poly-Lysine coated glass, Permanox, tissue culture plastic) if simply a liquid mixture (e.g. a transfection precipitate) is placed on the surface. These experiments were conducted by placing DNA mixtures onto the surfaces and staining with EtBr to visualize. Each type of DNA precipitate yields distinct pattern formations that may be representative of the type of precipitate formed on the surface. Permanox plastic has achieved the highest transduction efficiency and the greatest adherence of all surfaces tried so far. Tissue culture plastic has achieved nearly as high efficiency. Glass surfaces proved difficult because of hydrophobicity and difficulty of cell and DNA adherence. Glass coated with Poly-Lysine did not improve the characteristics of the plain glass surface to a great extent In fact, Poly-Lysine tended to cause spots to be difficult to form on the surface (liquids would not form a precise spot). The gene transduction vector used (e.g. plasmid DNA, lipid transfection vehicles, or viral vectors) can be but need not be dried on the surface of the array material. For example, inclusion of glycerol or trehalose, freezing of samples on the array, or arraying under conditions of high humidity can be used to spot array locations without drying of the samples.

The amount of spill-over of gene transduction to cells outside the intended bounds of the spot increases over time. Cells assayed after 1 day are typically well within the intended boundary, which is often visible under light microscopy as a thin dark line circling the perimeter of the spot Few cells expressing the marker gene outside the perimeter are usually visible. After two days, more cells outside the perimeter are visible, and after 3 days a significant number of cells can be seen outside the perimeter. Improved surface chemistry conditions may be able to contain spill-over more accurately and over longer periods of time.

Washing the DNA spot with buffer (e.g. PBS) can help to eliminate spill-over of the precipitate beyond the spot intended, but can also reduce the amount of DNA bound to the surface. In practice, the reduction in transfection efficiency from two 1 ml PBS washes was minimal while the reduction in spill-over was significant.

Any number of cell types can be used for this technology. Cells that readily adhere to the surface substrate and that are efficiently transduced by the vector chosen are the most easily adapted to the technology. Adherent cells should first be resuspended before adding to the slide. An even monolayer of cells is preferred for optimal expression of all spots in the array. Densely-packed cells may be advantageous for covering the entire slide and all positions of the array.

Cells in suspension are plated directly onto the array. Cells that settle onto the slide and adhere in a position over a particular spot (position) in the array will contact the genetic clone in the transduction vector. Cells contacting a specific clone will be transduced. The cells, now adhered to the slide surface and transduced with a gem, are allowed to express the gene (e.g. overnight incubation). 293 and 293T cells have been shown to work and any other type of cell type, cell line, or primary cell can also be used including, 293, 293T, QT6, HeLa, COS, CF2TH, CCC, CD4 cells, CD8 cells, Neurons, Astrocytes, Fibroblasts, Stem cells, Hematopoeitic stem cells, Progenitor cells, B-cells, and NK cells. Plant cells and plant cell-lines may also be used either as intact cells or as protoplasts with their cell walls removed, such as by enzymatic digestion.

Some cell types (e.g. primary cells) may be difficult to transduce with some vectors, and optimal conditions and gene transduction vehicles will have to be determined for these. Only routine experimentation should be required, however. The assay is not limited to existing cell types. Cells that are developed and prepared specially for this application (e.g. competent mammalian cells with greater transfection efficiency) can also be used. In addition, cell types with specially designed markers (e.g. signal cascade markers or transcription reporter genes) can also be used. For example, a cell line can be prepared that has a reporter gene (e.g. GFP) under the control of a MAPK-responsive promoter. When these cells are placed onto a cell-array, any gene on the chip that activates the MAPK pathway will activate the reporter, which can be easily detected.

Cells used for the cell-array can be manipulated prior to addition to the cell-array. For example, a vector that expresses a Tyr-kinase could be introduced into all the cells prior to addition of the cells to the cell-array. In this way, each cell would over-express two (or more) genes simultaneously—the single gene introduced into all the cells and a specific gene at the cell's position in the array. In this way, modification of proteins, cell pathways, and functions can be controlled, modified, and assayed for functional significance. For example, cells can be infected with a virus (e.g. Adenovirus or vaccinia virus) that expresses the Furin protease. Cells could also be transfected in bulk Once these cells are prepared, they can be placed onto the cell-array to express the gene at each position in the array. If the cell-array is designed, for instance, to contain 10,000 variants of the HIV Envelope protein, the effects of Furin on Envelope (cleavage and activation) can be determined using functional or chemical assays (e.g. fusion, ability to bind radiolabeled CD4, exposure of hidden epitopes that can be detected with antibodies). In one preferred embodiment, functional assays can be used to identify immunologic characteristics of a protein of an infectious agent For example, an array of HIV Envelope protein mutants can identify variants of the protein that are recognized by broadly cross-reactive neutraizing antibodies. The proteins encoded by such mutants could serve as vaccine candidates for eliciting a broad protective response.

Cells used on the cell-array need not be human in origin Cells from other species of primates, mammals, insects, plants, fish, birds, fungi or bacteria may be used. Genetic transduction mechanisms may need to be altered based on the type of cell used.

It is possible, for some applications of the cell-array, that cell recovery may be desired. In this case, surfaces that allow microdissection, physical isolation of cells, or laser-assisted recovery may be used to allow fine recovery of cells with a specific function or phenotype. This may be especially useful when screening diverse pools of cells with unique qualities (eg. B-cells, T-cells, hybridomas). Cells with a desired phenotype can be used for disease models or for iterative identification of genes along a pathway.

Cell-based assays are important for functional screening of genes to identify new drug targets and gene therapeutics. In a cell-based assay, cells are transduced with a gene, measured for interaction with a probe, and/or followed by determining changes in cellular behavior or phenotype. For example, a proliferation assay can be used to determine genes that trigger proliferation and that might be causal to a certain cancer.

The cells on the cell-array, living and overexpressing defined functional proteins at very high levels, can be assayed using any number of techniques. Once transduction has occurred and the cells have taken in the cloned cDNA, the effect of the expression of the cDNA can be observed. It is at this stage that perhaps the greatest value of the cell-array arises. Those skilled in the art will know many ways to screen expressed sequences for the functions they desire to investigate.

Hundreds of assays have already been adapted to the standard 1×3 inch slide format, and a variety of parameters can be measured using automated detection systems that have already been developed. Some of the many functional and biochemical assays that could be utilized include, reporter gene expression, cell proliferation (e.g. agar overlay), phenotypic change, RNA transcription, cell migration, capillary formation, intracellular localization, differentiation, enzyme activity, cytotoxicity, infection, fusion, and binding. All are known, per se, to persons skilled in the art.

Currently available fluorescent detection systems can detect a fluorescently labeled probe on a 1×3 inch slide in 1 minute at 5-10 um resolution. Software for reading and interpreting this data has also been developed by third parties for analyzing standard gene-based arrays. By analyzing the resultant 1 billion data points, we can rapidly identify those few cells that contain the probe of interest or that display the desired phenotypic change. Other labeling techniques, such as radioactivity, could also be employed.

Any and all detection methods can be utilized. Mechanical, optical, and laser array reading possibilities that currently exist and future detection technologies that can be created are capable of detecting the different signals of output assays. In addition, new detection methods that have unique applications to our technology, such as intracellular imaging, may be developed. The cell-array is designed to be amenable to assay and detection using any existing or future detection techniques that can be applied to cells, arrays, or slides. When a spot with a desired property (i.e. signal) is detected, its position in the array makes identification of the gene that caused the signal trivial. A non-exhaustive list of detection technologies includes, fluorescent labeling, radiolabeling, colorimetric assays, immunohistochemistry, optical detection, cell staining, time-resolved fluorescence spectroscopy for real-time binding, fluorescence microscopy, spectroscopy, DNA/RNA hybridization (e.g. with cellular DNA/RNA), in situ hybridization, scanning probe potentiometry, automated intracellular imaging, surface plasmon resonance, confocal microscopy (e.g. automated), atomic force microscopy, miniaturized electronic biosensors (e.g. at each array position), scanning electron microscopy (e.g. automated), SELDI (surface-enhanced laser desorption/ionization), MALDI TOF (Matrix-assisted laser desorption/ionization time-of-flight), and other mass spectrometry-based detection methods. Each of these are known, per se, to persons skilled in the art.

Both intracellular and extracellular proteins can be assayed. Extracellular proteins will be directly accessible to assay and detection. Intracellular proteins can be accessed using any number of standard mechanisms, including the use of membrane-permeable substrates. Detergents are also readily available that can make all intracellular proteins accessible. Detergents range from strong ionic detergents (e.g. SDS) that could disrupt all cells on the slide to very mild, non-ionic detergents (e.g. digitonin) or porin proteins (e.g. Streptolysin-O) that merely create small pores in the cell membrane. Cells can be fixed (e.g. with formaldehyde or methanol), stained (e.g. standard immunohistochemistry), probed (e.g. in situ hybridization), or assayed (e.g. for transcription-driven markers) as needed, either in a living state or in a fixed state.

The cell-array can take any physical form that can accommodate an array on which living cells will be placed. In the envisioned form, the cell-array will physically be composed of a plastic slide that measure 1×3 inches and is about 1/16 inch thick Such a slide can adapt to any number of currently available readers, adapters, techniques, and devices. However, other modalities, such as 96-well sized plates, can also be used. Kits including such arrays may be produced.

The cell-array is particularly suitable for robotics and automation under the control of a computer. High-throughput, robotic, biomaterial-dispensing systems are available to allow precise and accurate addressability of substrates during array “printing” of nucleic acids. Most of the requisite engineering has already been performed in the course of building standard gene arrays used by the genomics industry. For example, gene arrays (spotters) are commercially available and can array 10,000-40,000 spots on a standard 1×3 inch slide. A 40,000 feature array can be composed of a 200×200 matrix Currently available machines are very capable of producing tens of thousands of spots per arrayers and the technology is improving at a very rapid pace. Current array technologies include pin-based arrays, ink-jet based arrays, photolithography, and piezoelectric arrayers, any of which could be used to produce an array on a cell-array. The use of such arrayers for production of a cell-array capable of gene transduction is a further aspect of the invention.

At a feature size of 50-200 microns (spot diameter), such an array readily fits on a 1×3 inch glass slide. Since many cells range in diameter from 1 μm to 10 μm, each spot in an array can be designed to transduce anywhere between 25 and 40,000 cells.

The cell-array is differentiated, inter alia, from other forms of “bio-chips” by the functional expression of proteins, the physical architecture, the structural integrity of proteins immobilized on the surface, and the ability to measure a variety of in vitro, in situ, and functional assays. Uses for the cell-array include protein discovery, protein profiling, structure determination, activity measurements, as well as the assessment of protein-protein and protein-small molecule interactions.

Cell-arrays allow for the rapid identification and characterization of proteins, including the small bioactive peptides and rare proteins missed with 2-D gel technologies. Identification and functional characterization of proteins that are expressed in disease state can now be achieved Cell-array libraries can also be used to screen for cells that express specific therapeutic proteins of interest.

The present invention can be used to produce and characterize proteins of all types. Even complex proteins such as G-protein Coupled Receptor ion channels, and HIV Envelope can be produced with ease. In one example, a human gene library can be used to express random proteins that will still accurately express these complex (and the other simple) proteins. In another example, a mutation library of a single type of protein (e.g. a random mutagenesis of HIV Envelope or a GPCR) can be arrayed and assayed for function or other phenotypic characteristics.

Expression libraries may also be used to create and isolate cell lines that express validated protein targets of interest. Once the appropriate cell line expressing the protein target of interest has been isolated, the cell-array platform can be used to apply a variety of drug discovery techniques to identify lead candidates for drugs that may interact with this target.

Using automated detection technologies (e.g. in situ hybridization, DNA/RNA hybridization), the cell-array is capable of detecting changes in the expression or localization of any protein The protein of interest is not necessarily limited to the protein encoded by the transduced gene. In other words, the characteristics of one protein or gene can be monitored in the presence (or absence) of every other protein introduced using the array.

An alternative application of the cell-array methodology is to produce proteins in situ on a chip. mRNA can be captured, synthesized, or produced with or without a cell at a specific location on the chip. With the mRNA at a specific location, in vitro translation can be initiated using standard protocols to produce proteins or peptides directly at the site of mRNA location. The protein synthesized can be bonded to the same site of synthesis using cell-array surface chemistry, new chemistries, or affinity-tags embedded in the proteins themselves (e.g. epitope tags and antibody-coated slides).

Invasion of cells (human or non-human) by infectious diseases requires a cellular receptor. These receptors are often ideal candidates for drug intervention, and the infectious disease protein that interacts with these receptors is often an ideal candidate for vaccine development. Identification of these receptors can be accomplished using the cell-array by allowing live infectious agents to invade living cells. If the cells are not normally permissive for entry, then expression of the correct protein (receptor) will allow entry of the agent. If the cells are naturally permissive for entry, then elimination of expression of critical genes (e.g. using antisense cell-arrays) will disable the agent from entering or replicating in the cell. The genes identified may be involved in entry or in post-entry events such as assembly, replication, or release from the cell.

Through the sequential identification of multiple genes involved in the entry and replication of an infectious agent, an entire pathway can be mapped. In this iterative fashion, completely non-permissive cells (e.g. murine cells) can be made permissive for steps in infectious agent (e.g. HIV) invasion (e.g. entry, nuclear transport, transcription, assembly, budding, etc.). In addition, alternative pathways of replication or blockage of replication can be functionally mapped.

Cell-arrays have application to numerous infectious agents, such as, HIV, hepatitis strains, ebola, other viral strains, tuberculosis, N. meningitis, and other bacteria strains. As well as viruses, retroviruses, prior-caused disease, metabolic disorders and other conditions.

The present invention permits the discovery of a gene, the discovery of the function of that gene, and measurement of the functional consequences of alterations in the gene. Massively parallel screening of function, as is now provided, automates the measurement of thousands of physical and chemical characteristics of a selected organism's genes at different times of the organism's life cycle by profiling protein expression and cellular phenotype. Versatile, distinct assays can be used for functional screening of morphology, cell shape, capillary formation, invasion, motility, localization of expressed reporter genes, NO production, growth factors, enzyme substrates, and other factors.

Cell-arrays can be used to identify and characterize proteins that confer resistance to chemotherapeutic agents in tumor cells, control the growth or formation of specific cell or tissue types (such as nerve cells, immune cells, or other cell types), control immune cell function (e.g. antibody production), and affect tumor cell formation.

Other applications include rapid analysis of genetically modified plants, glycosylation assay development, peptide binding assays, antigen capture from natural killer cells, beta-amyloid peptide assay, identification of substrates for proteases, capture of cytokines by orphan receptors, retentate mapping of Mycoplasm to establish phylogeny, assay for drug effect on a HeLa cell marker protein, DNA-protein interactions, quantitation of bioactive peptides, actin-binding venom peptides, identification of a drug target protein, prostate cancer multi-antigen immunoassay, assay for nicotinic acetylcholine receptor activity, cell-cell interactions (e.g. sperm-egg fusion), localization of genes to intracellular compartments (e.g. GFP-tagged genes, follow post-translational processing for all proteins), over- or under-production of protein on a pathway (e.g. vitamins, amino acids) to find genes that regulate metabolic pathways, and other relationships.

Small-molecule drugs act on proteins. Knowledge of a protein's structure and how structure encodes function is crucial to the rational design and optimization of candidate drugs. Structure/function studies help to identify the site on the protein that should be targeted by a drug. This information can be gleaned only from the direct study of proteins. Structure/function studies, as with protein expression work, currently ale performed principally with decades old technology. Higher-order structural information is critical to drug discovery and it can only be determined by investigating proteins directly. The cell-array technology can control and identify the precise form—splice variant and/or post-translational modification—of a protein that confers a specific function. Examples of protein characterization programs include mapping of protein phosphorylation sites, B-lactoglobulin peptide mapping and protein ID, protein purification and protease mapping, peptide mapping of proteases and secretases, mapping of phosphorylation sites on proteins, protein glycosylation assays (N— and O-linked carbohydrates), mapping of protease cleavage sites (e.g. Furin sites), mapping of protein sulfation sites and their effect on function, identification of DNA, RNA, or protein modifiers by using detection substrates (e.g. restriction enzymes, phosphorylation), and other things.

Mutations in complex proteins can be screened at a high rate of speed for phenotype or function using the cell-array. For example, random mutants of complex proteins such as HIV Envelope and G-protein Coupled Receptors can be generated and screened on a customized cell-array. Function, structure, and reactivity (e.g. MAbs) can be analyzed and only mutants with desired characteristics need be isolated or sequenced.

Proteins act through concerted pathways, or networks, rather than in isolation. Many biological pathways are of a cascade nature, where the initiating action kicks off multiple second-order actions, each of which, in turn, initiates multiple third-order actions. These pathways typically contain key regulatory junctions, where entire pathways may be turned on or off. It is critical to map pathways in order to identify the optimal point of intervention, such as at the initiating signal or a key regulatory juncture, e.g. of a pro-inflammatory pathway for an anti-inflammatory drug candidate.

Pathway and network mapping studies allow one to establish the relationships between the fundamental biological commands used in the cell. Gene expression studies can identify all of the commands used in a cell's biological program and how often each one is used, but little about how those instructions code for function. There haw been few fundamental advances in network mapping technology over the past 15 or so years. Up to now, mapping a pathway has taken years and even decades. New technologies such as yeast-2-hybrid are fundamentally speeding this mapping, but are limited in fundamental ways: mammalian pathways can not be mapped, extracellular interactions such as ligand-receptor binding can not be mapped, and modified forms of proteins (e.g. phosphorylated and glycosylated) can not be assayed. Cell-arrays uniquely enable proteins to be processed from a variety of cells and species in order to determine the pathways and networks within which they operate. A potential ligand can be assayed for interaction with every other protein expressed in the human genome, both intracellular and extracellular. With very minor modification of the cell-array, we can control nearly all forms of protein modification (both known and unknown) to determine if post-translational modification of a protein is required for interaction with its ligands.

It is important to note that a fundamental advance inherent to the cell-array is the ability to map extracellular functional pathways of the human proteome. Since approximately 50% of drugs are targeted to extracellular, membrane-embedded receptors (e.g,. GPCRs and ion channels), the current efforts to map human protein interactions are lacking an efficient enabling technology. Cell-arrays and other aspects of this invention permits one to map entire protein-protein intracellular and extracellular functional pathways, find new proteins interacting with other new and known proteins, and eliminate potential targets rapidly because they interact with multiple signaling pathways, thus identifying the protein as a less desirable target.

The interactions of proteins can also be assessed by co-expressing proteins in the same cell. For example, every cell added can be transduced with a single specific gene such as a Tyrosine kinase (e.g. by transfection in bulk, creation of a stable cell line, or by infection with a designed virus). Alternatively, every location in the array can have this gene for transduction. When each spot in the array expresses a different gene (in addition to the first one), the result will be an array that has two genes expressed in every cell in the array—one defined (e.g. the Tyr-kinase) and the other specific to the position in the array. The interaction of the two proteins can be assessed using visual colocation, transcriptional reporting, or other detection techniques. Alternatively, the functional effect of the coexpression can be monitored using any number of functional assays. Comparison of identical arrays, only with or without the constant gene, allows controlled experiments to be run. If the modifying (constant) gene encodes for a protein modifying enzyme (e.g. kinase, phosphatase, glycosidase, etc.), the post-translational regulation and modification of proteins can be assayed.

The interaction of proteins (and small molecules) can also be applied using the cell array for the purpose of identifying unwanted interactions. For example, many therapeutic proteins, antibodies, and chemicals interact with proteins other than the targets they are intended to interact with Using the cell-array, these unwanted targets can be identified in advanced and correlated with clinical side-effects, toxicity, or bioavailability. In this way, the cell-array can enhance the probability of late-stage clinical success.

Cell-arrays can express tens of thousands of proteins simultaneously, providing an efficient substrate for determining what antibodies are currently active in the human body (the human “immunome”). A human cell-array enables the targets of auto-antigenic antibodies to be determined. Cell-arrays from other species allows the diagnostic ability to detect antibodies directed against proteins of other, potentially pathogenic, organisms. Quantitative description of the antibodies present in an individual may make an important diagnostic tool to describe what happens in an immune system over time, at stasis, when perturbed by infections (e.g. HIV, rhinovirus), or when responding to cancer, a vaccine, etc. Autoimmune disorders (e.g. arthritis, lupus, etc.) may be particularly amenable to detection using the cell-array with a human gene library, and diagnostics may be a key market for this application. Cells can be permeabilized to detect intracellular and extracellular proteins.

The cell-array system can be used for the selection of peptides, proteins, and small molecules with desired properties. Cell-arrays and libraries constructed from human cells and tissues allow analysis of protein:protein, enzyme:substrate, and drug:protein interactions. Molecules can bind to or cause a functional response and be detected using the cell-array. Targets may be involved in a variety of important biological processes, including the production of proteins that function in central nervous system function; function in cell growth and differentiation; regulate immune cell function; control metabolic functions, such as glucose metabolism; relate to viral infection; and affect other key biological processes.

Cell-array technology in accordance with the invention facilitates the discovery and characterization of novel human genes, which might otherwise be difficult to identify using alternative approaches. The protocols can activate and isolate specific types of protein families, such as receptors and secreted proteins, that may have particular relevance to the drug discovery and development process. Of special note, cell-arrays can be used to screen for proteins that reside in the membrane surface of a cell, commonly referred to as integral membrane proteins. This class of proteins has accounted for approximately 50% of the drug targets that have been identified and utilized by the pharmaceutical industry to date.

Mutant or diseased cells can also be screened on cell-arrays for alteration of function or phenotype that may indicate links to disease or cures for a phenotype/disease that may be achieved directly through gene therapy, anti-sense therapy, peptide therapy, or protein therapeutics or through small molecules. The ability to screen for phenotypic and functional changes in cells that relate directly to disease is a fundamental approach for identifying and validating important proteins and genes in the context of disease.

Biologists can screen proteins on the chip for interaction with organic molecules, non-organic molecules, peptides, proteins, DNA, RNA, metal ions, lipids, membranes, whole families of receptors, entire classes of enzymes, complete categories of antibodies, whole cells, antibodies, and many other species.

The effects of candidate drugs intended to reverse a disease process, and the determination of the degree to which this objective is achieved free of adverse side effects on cells or interaction with other proteins is another aspect of the invention. In addition, cell arrays are not limited to detecting interactions with cellular proteins. They can be used to screen any substance contained within, on the outside, or released by a cell, including DNA, RNA, ions, organic molecules, enzyme cofactors, organelles, membranes, peptides, proteins, and other species.

In another embodiment, the cell-array can be used to identify substrates for drug targets. For example, starting with a protease target, the protease can be expressed in every cell on the cell-array which then co-expresses potential substrates or modified substrates. New substrates that are cleaved by the protease or mutant substrates that are resistant to the protease can then be identified and used for drug development.

Cell-arrays and other embodiments of the invention can be used to identify and define the proteome, the array of proteins expressed in a human cell. With each cell in the cell-array expressing a defined gene, the effects of that gene on the rest of cell's proteome can be defined. For this purpose, special chip surfaces may need to be utilized that allow gene transduction and cell growth but that also allow capture of proteins via mass spectrometry. Techniques such as SELDI (surface enhanced laser desorption/ionization) that can ionize specific spots within an array could be suited for analysis of the proteome. Applying such an analysis across an entire cell-array expressing the human genome would allow a researcher to define how each gene in the human genome effects every other protein in a cell.

The cell-array can be designed in a manner that allows microfluidic channels to be incorporated into the chip. In this manner, infusions of molecules directly to cells of interest can be discretely controlled. Alternatively, proteins released from discrete subsets of cells could be harvested and analyzed. In one embodiment, cells overlaying a microfluidic channel could receive a continuous stream of reagents, such as chemicals, antibodies, or potential ligands, that could then be used to detect a cellular response.

Cell-arrays enable scientists to conduct differential diagnosis of the immunome, the complete set of immunologic targets in a human. Protein expression of the human genome will enable the diagnosis of immune and inflammatory diseases that are directed to self-antigens. Rapid identification of multiple protein disease markers simultaneously represents a tremendous improvement over existing assays. Single protein disease markers, such as PSA for prostate cancer or CA125 for ovarian cancer, have limited reliability for early detection, and their use remains controversial. Some examples of cell-array uses for diagnostics include biomarker discovery, schizophrenia diagnostic markers, kidney stone disease marker, protein profiling of cell lysates, validation of protein markers, prostate cancer markers, bladder cancer markers from urine, toxicology correlation of drug use with immunological response, expression profiling, for target identification and validation, toxicology profiling, for drug lead selection, diagnostic evaluation, for patient management, disease management, for therapy selection, and others.

Because the cell-array and other aspects of the invention can express an entire genome simultaneously, small molecules can be screened against the proteins from the genome to identify reactions. In this manner, purified monoclonal antibodies and small-molecules (e.g. organic drugs) can be identified that target proteins of specific structures or phenotypes of defined function, even without knowing the precise target of interest.

For example, a random, purified monoclonal antibody from a defined hybridoma clone can be used to screen a cell-array. The same could be done for a chemically pure small-molecule. The protein that the antibody reacts with can then be defined. Monoclonal antibodies should react specifically with only one protein. This may be useful if antibodies (or small molecules) have effects of interest, but their targets are not known. Moreover, if a random antibody or chemical binds to a small number of targets on the cell-array (ideally a single target), then the specificity of that antibody or chemical is defined. Screening a large number of purified monoclonal antibodies or chemically pure small-molecules will enable the development of a library of antibodies/chemicals that have already been pre-screened for the specificity desired. Moreover, if these antibodies or chemicals are also pre-screened for their toxicity, bioavailability, etc., a library of compounds will arise that has known specificity and that are pre-screened to be better compounds for drug development—i.e. a library of lead compounds to defined targets. Targets of complex nature (e.g. membrane receptors, glycosylated proteins) are particularly amenable to the cell-array.

A large library can be built even before specific targets are linked to specific diseases or phenotypes. For example, a biotechnology company may discover that a new gene (X) is involved in cancer. Rather than begin screening for small-molecule lead compounds or antibodies to that new gene, a compound and/or antibody specific to that gene that has already been screened for desirable characteristics will be identified for use. In this manner, the early stages of drug development can be hastened and better molecules for human application (e.g. toxicity, bioavailability) can enter drug discovery.

In another application to achieve a similar result, purified panels of monoclonal antibodies (to unknown epitopes or target proteins) can be spotted on a customized cell-array. The chip can then be screened against proteins of interest in order to identify which, if any, of the antibodies on the chip bind the protein of interest MAb supernatants can be spotted on the cell-array for this purpose. Alternatively, genes encoding for MAbs (e.g. random and mutagenized) can be used for screening purposes. Completely human MAbs can be generated and isolated in this manner. Similar results can potentially be obtained for small-molecule compounds if they are spotted directly on the chip.

Antibody and T-cell receptor responses can also be generated in a similar way if genes encoding for proteins or epitopes are arrayed on the cell-array and then hybridomas or T-cells are used as the cells on the array. The cells will be transduced with the protein and will respond appropriately by producing the protein. If the cell also produces a T-cell receptor or antibody that reacts with the expressed protein, it can be detected using a number of techniques. The cells may be recovered by laser ablation, dissection, or otherwise.

Cell-array libraries can be used to search for cells that exhibit specific biological properties. When a cell with a desired feature is detected, we can rapidly and directly associate this specific characteristic with the expressed gene by its location in the array. One strategy avoids the less efficient extrapolation of gene function from gene sequence that has, up to now, been the industry paradigm. The cell-array can be used with cells from a wide variety of species that are of commercial interest. Cell lines, specially prepared cell lines (e.g. with gene markers or transcriptional signals), and primary cells can be used.

The cell-array is an easy to use platform for target discovery and validation. The cell-array can be shipped to scientists ready to use and can be stored for months or years in a standard laboratory freezer. Any number of cell types, including primary cells, can be used on the chip, and achieving expression of every gene on the chip can be accomplished overnight. The cell-array technology offers a number of superior characteristics, these include simultaneous expression of thousands or tens of thousands of genes, expression libraries can include an entire organism's genome or tens of thousands of mutants of a single gene that could then be selected for function, assays can be performed within days (most assays will typically take 2-4 days, but an assay can be performed in as little as one day), multiple chips can be processed simultaneously, allowing comparative treatments and conditions, and others.

Protein expression libraries express tissue-specific and rarely expressed genes as well as abundantly expressed genes at comparable frequencies. As a result, significant biases toward genes that are ordinarily expressed at high levels or in many tissues can be minimized or avoided. Libraries used can ensure significant coverage of the entire genome, including rarely expressed genes encoding key biological regulators, which are believed to be valuable drug targets or therapeutic candidates.

The present invention is compatible with a variety of different biological model systems, or assays, including biochemical, cellular or even animal assays. In addition, libraries may be created from a variety of cell types, including human, animal, plant, or prokaryotic cells. Cell-arrays can be used to generate cell lines that express activated genes at high levels. These cell lines can be used to produce large quantities of proteins for biochemical studies, in cell-based assays for screening therapeutic compounds, or for functional genomics studies. In addition, genes may be permanently or temporarily expressed, depending on the goals of the research project. Cell-arrays can also be used to activate genes in a manner that does not require the isolation and cloning of individual genes or the use of gene sequence information.

Post-translational modifications of proteins can be monitored and controlled using the cell-array. For example, the functional form of a protein can be recovered from the cell-array to ascertain any post-translational modification Even further, cells that are co-expressing genes that affect post-translational modification can be used to control and measure the function of proteins when they are modified. For example, every cell used in a cell-array can be made to express a Tyr-kinase just before the cells are added to the cell-array. In another example, the modifying gene does not need to be known—a single, even random, gene can be over-expressed in every cell just before the cells are added to the cell-array. Defined functional effects of the co-expression can then be measured.

Many proteins are modified after they are made. These modifications—the addition of a phosphate or complex carbohydrate, for example—often critically affect protein function. Many proteins are only slightly active to completely inactive until they are appropriately modified. Gene expression studies indicate nothing about post-translational modifications.

The breakdown products of proteins often have unique functions in their own right For instance, the well-known anti-angiogenesis drug candidates, angiostatin and endostatin are each fragments of other proteins—plasminogen and collagen Type XVIII, respectively. Gene expression studies cannot identify potentially bioactive fragments of proteins; only protein expression studies can make this distinction. Gene expression studies do not provide a complete picture of normal or disease biology, but merely the outline of the cell's biological program Protein expression studies complement gene expression information for multiple reasons. Thus, mRNA and protein levels are not always correlated and splice variants of genes can produce multiple forms of proteins. Protein expression studies can identify which splice variants are being made, and whether or not the splice variant produced by a given gene changes in disease. Importantly, these variants can be identified after the phenotype of interest is uncovered, saving time by reducing the human proteome an order of magnitude to the size of the genome.

The cellular architecture of the cell-array offers a number of advantageous attributes including stability of expressed proteins, ease of manufacture, ease of detection using standard assays, ability to control binding and assay conditions, high packing density for massively parallel protein expression, and structurally intact conformation and orientation of proteins.

The use of cell-arrays, and other aspects of the invention for the identification of protein:protein interactions is an attractive alternative to traditional yeast two-hybrid systems because they can utilize proteins derived from any type of organism—human, microbial, plant, etc.—and the technology can express the entire, structurally intact version of membrane-bound proteins and receptors. Cell-arrays have numerous advantages over the commonly employed protein separation/purification technology (giant 2-D gel electrophoresis), which is a decades-old technology. In particular, cell-arrays are rapid, reproducible, and can be used to probe the function of even very rarely expressed genes. They enable follow-up investigations, such as structure/function studies, to be performed directly on chip-bound proteins. Giant 2-D gels, on the other hand, are slow, not terribly reproducible, require large sample sizes, and require significant further purification work (liquid chromatography or some equivalent means) before proteins can either be identified or investigated further. Moreover, the array format of cell-arrays, coupled with different libraries representing different types of genes, allows unparalleled flexibility in determining gene function. A single chip can identify and determine the function of more proteins than can be separated on a single giant 2-D gel. Cell-array libraries can also be created from numerous sources, and can identify genes that are lost on giant 2-D gels due to size limitations and problems with handling membrane-spanning regions of proteins.

Cell-arrays and other embodiments of the invention have a diverse range of applications for understanding the basic functions of the human genome. Several diseases will be immediately amenable to drug development using the cell-array. We can isolate hundreds of proteins directly associated with the cellular phenotype that causes major chronic and acute diseases. Examples of functional pathways that can be studied with the cell-array and the associated disease applications include pathways of cellular proliferation (Cancer), personalized vaccines (Non-Hodgkin's lymphoma), cell lysis (antimicrobial peptides), stimulation of hematopoietic growth factors (bone marrow transplants), differentiation of cells, cell proliferation and oncogene identification, and fat deposition increase or decrease (obesity). Other important diseases include asthma/allergy, autoimmunity, cardiovascular disease, diabetes, osteoporosis, osteoarritis, obesity, rheumatoid arthritis, transplant rejection, tumor growth programs, viral infectious agents, bacterial infectious agents, fungal infectious agents, and metabolic profiling.

When applied to crop production, functional genomics can enhance the nutritional content of foods, select for enhanced phenotypes, reduce the effects of farming on the environment, and develop foods that can enhance food production. Arabidopsis thalania, rice, corn, and soy will be prime agricultural targets for functional genomics. Arabidopsis is a useful model organism because it is related to soybeans, cotton, vegetables and oil seed crops. Rice is an important target and model organism because it is one of the world's most important grains and commodity crops, and it is closely related to corn, wheat, barley, sugarcane, oats and rye.

One of the unique attributes of Protein Chip Expression technology is the ability to rapidly identity antibodies that are differentially expressed in immune-related diseases. We will exploit this capability in proof-of-principle studies to identify novel auto-antigens that are targeted in human immune disorders such as arthritis, asthma, and allergy. The applications that this capability enables is two-fold: 1) to identify and patent novel disease markers as diagnostics, differential diagnostics and patient management tools; and 2) to establish the cell-array as the platform technology to perform diagnostic testing with novel protein markers, which would translate into chip sales.

One example of the application of a diagnostic use for the cell-array involves lymphoma. The cell-array can be used first to identify what B-cells have mutated based on the over-production of a specific antibody and the reactivity of antibodies produced by that B-cell to a protein on the cell-array. Next, a peptide antigen directed to that antibody can be designed and used as a radiological marker or drug (e.g. radiolabeled or linked with a toxic gene).

Viral vectors allow the expression of known and unknown genes in a large range of host organisms and cell types in order to determine gene function (functional genomics), and can enable the expression of genes in cells used for the production of therapeutics (biomanufacturing).

Retrovirus-based technologies can introduce a large library of genes or gene fragments into cells of all types. Each retrovirus can be designed to encode a specific gene and each virus with a unique gene can be placed on the cell-array. A defined virus is then used to infect a cell of interest in order to over-express a specific gene. The methods described herein enable the creation of a library of such retroviruses and placing tens of thousands of them on a 1×3 inch glass slide. Alternatively, some libraries are commercially available, either in arrayed or non-arrayed format.

One advantage of using retroviruses is that once a gene or function is identified, the retroviral probe that caused the desired phenotypic change can be transferred to other cells, including animal models, and used in further development Retroviruses are also capable of infecting many cell types, including cell lines, primary cells, and non-dividing cells.

Adenoviral vectors (Ad) are a commonly used gene delivery system for gene transduction into human cells and tissues because of their high transduction efficiency. The adenoviral vector carries the transgene into the target cell, but does not integrate it into the target cell genome. Arrayed adenoviral libraries in a cell-array format, as described herein, could enable high levels of expression in cells and provide a high gene transduction rate at each spot on an array. The greatest benefit of an arrayed library format is the ability to perform versatile, functional assays with a wide variety of human cell types, including primary cells. Adenoviral vectors have advantages including broad host range and low pathogenicity. Adenoviruses can infect a broad range of mammalian cells and therefore permit the expression of recombinant proteins in most mammalian cell lines and tissues and infection and expression of genes in both replicative and non-replicative cells. Additionally, adenoviruses can infect virtually all cell types with the exception of some lymphoid cells. This allows for a direct comparison of results obtained with transformed cell lines and primary cells. They replicate efficiently to high titers. The Ad system allows production of 10¹⁰ to 10¹¹ VP/nL which can be concentrated up to 10¹³ VP/nL. This feature makes it a very good vector system for large-scale applications. Helper-independent Ad can accommodate up to 7.5 kb of foreign DNA. To provide additional cloning space, the E1 and E3 early regions of Ad can be deleted. Additionally, Ad can normally encapsidate a viral DNA molecule slightly bigger than the normal DNA (105%). These combined features allow for the insertion of an expression cassette containing a gene or multiple genes of up to 7.5 kb into one recombinant Ad. The Ad expression system can be designed to express multiple genes in the same cell line or tissue. The Ad can contain two genes in a double expression cassette of the Ad transfer vectors. Alternatively, using different recombinant viruses each expressing a different protein, a co-infection of the desired cell lines can be performed. Determining the MOI ratio of the different recombinant viruses will provide the proper relative co-expression of the recombinant proteins. Moreover, there is no insertional mutagenesis; Ad remains epichromosomal in all known cells except eggs and therefore does not interfere with other host genes. The integration of only one copy of virus in zona-free eggs is a better system to produce transgenic animals with specific characteristics. The Ad vector system uses a human virus as a vector and human cells as a host. It therefore provides the ideal environment for proper folding and exact post-translational modifications of human proteins.

### EXAMPLES

The present invention, as embodied in cell-arrays, integrates protein biochemistry with advanced materials science and microfabrication to create a miniaturized chip containing high-density arrays of functional proteins to quickly and accurately correlate protein function with genetic composition. The cell-array technology has been constructed from a single-use, disposable plastic slide expressing functional and structurally intact proteins in cells that are bonded to the surface. The primary components of the technology have now been demonstrated to function in accordance with the invention.

The library is contained with a gene transduction vehicle that will allow the vector to adhere to the slide but to also transduce the cell. The current technology has been enabled, inter alia, using a cDNA construct expressing an easily measured molecular marker (Green Fluorescent Protein (GFP) in a pcDNA3 vector with a CMV promoter). However, any construct, plasmid, gene, or gene fragment could have been used as well.

A present embodiment has been prepared using lipid-based transfection vectors Lipofectamine, Lipofectamine Plus, Lipofectamine 2000, and Gene Porter™. Calcium-phosphate has also been used. The precipitate formed by each of these methodologies was allowed to air-dry on a surface before placing cells on the surface. The liquid precipitate mixture is also placed on the surface, allowing the precipitate to form and settle on the surface. The rest of the liquid is washed from the transfection precipitate (i.e. no air dry step). If the liquid precipitate is left on the slide for sufficient time (e.g. 1 h), the precipitate settles, adheres to the surface sufficiently to withstand washing, and can then be used directly for gene transduction without a drying step. The precipitate can also be allowed to adhere to the surface, the media is replaced, and then cells added. A spot of diluted Lipofectamine can also be placed directly on the slide whereupon a spot of diluted DNA is placed on top. This methodology allows an effective precipitate to form directly at the array position of interest rather than being formed in a tube prior to placement on the surface.

Gene expression using this methodology increases significantly over time. Cells assayed 2 days following gene transduction can express double or triple the amount of marker gene than cells assayed the day after gene transduction. Cells assayed 3 days following gene transduction express incrementally more (e.g. 20-40%) marker. This increase in gene transduction efficiency, however, is offset in part by increased spill-over of expression outside the intended bounds of gene transduction.

Optimem media was used for transfection precipitate formation although other medias may be employed. 10% DMEM with 1% Pen-Strep was used for growth of cells because of its wide-spread use for standard tissue culture growth. Other media types wil work similarly, although some types may yield different efficiency. Antibiotics and serum may have a particularly strong effect on gene transduction efficiency.

Cell arrays in accordance with certain preferred embodiments can be demonstrated. A monolayer of cells, such as HEK-293T cells, all of which are identical can be deposited on a substrate surface. The array may be such as to have a marker gene, e.g. Green Fluorescence Protein, that has been transduced into a defined subset of the cells. Clearly defined fluorescence gives a visual indication of the controlled demarcation of gene transduction.

While optimal conditions for gene expression will be determined for each particular circumstance and system, as an example, a protocol follows that has been used for prototype development. Variations are included in the other sections discussing each component of the technology. The current protocol for the use of the cell-array technology is performed, in one exemplary embodiment, as follows:

a. 100 μl Optimem media was combined with 1 μg DNA (pcDNA3-GFP) and 6 μl Plus reagent (art of the Lipofectamine Plus commercial reagent package from Life Technologies)

b. In a separate tube, 100 μl Optimem media was combined with 4 μl Lipofectamine

c. Both tubes were allowed to incubate at room temperature for 15 minutes

d. The DNA mixture was then added to the Lipofectamine mixture with gentle vortexing

e. The tube was allowed to incubate at room temperature for 15 minutes

f. 10 μl of the mixture was placed as a spot on a Permanox cell-culture slide forming a spot of approximately 3 mm diameter

g. The spot was allowed to air-dry in a sterile tissue culture environment without a lid and at room temperature for approximately 2 hours until the liquid had evaporated and dry residue was visible where the liquid mixture had been placed

h. The well was washed twice with 1 ml of PBS

i. 2×10⁵ 293T cells were resuspended in 0.5 ml 10% DMEM media, added to the well (the size of a 24-well), and allowed to settle onto the surface

j. Cells were incubated 1-2 days at 37° C. and allowed to express the gene

k. Gene expression was monitored using an inverted epi-fluorescent microscope with a light filter that allowed detection of GFP expression

Cells on a cell-array made in this way can be assayed. Spots representing cells transduced with the pcDNA3-GFP vector and distinguished from dark spaces between the spots, which contain cells that have not been transduced (visible under normal white light illumination). The vector chosen represents a convenient marker, but any plasmid or gene could have been chosen for any or all the spots in the array.

Spots have been formed on a surface ranging from 0.1 μl to 20 μl, thus achieving the lowest limit possible with manual pipetting. In each case, cells were observed within spots expressing the marker gene (GFP). Spots of decreasing size achieved diminished gene transduction frequency (1-20%), while the larger spots (10-20 μl ) could achieve gene transduction frequencies of over 50%. This response is likely a result of the amount of precipitate able to be placed within a spot (smaller drops of transfection mixture have less volume of precipitate).

Methods for simplifying the automated placement of transfection mixture have been developed. Rather than mixing lipid with DNA to obtain a precipitate prior to addition to cells or to a spot on a slide (a normal transfection protocol), a spot of diluted Lipofectamine was placed on the slide and then a spot of DNA was subsequently placed on top. This methodology allows an effective precipitate to form directly at the array position of interest, and avoids the problem of the precipitate not staying well mixed in solution during a prolonged arraying procedure. This methodology thus represents one possible mechanism for automated production of a cell-array array. A modified gene arrayer might be necessary for producing cell-arrays using such a technique. For example, an arrayer with a dual-pin slide could first drop Lipofectamine onto a slide, then drop the DNA onto the first drop. Alternatively, the slide may first be coated with a Lipofectamine layer. Alternatively, spots of DNA can be arrayed on a slide and then lipid-based transfectant can be placed over the DNA to form a precipitate at the location of the DNA.

A second method for high throughput transfection spotting has also been enabled. A lipid transfection mixture is prepared using only the lipid (e.g. Lipofectamine) and media. This was spotted onto a slide and allowed to dry. DNA-containing transfection mixture was then placed on top of the dried lipid and allowed to precipitate and dry at the spot of interest In this way, an entire slide can be coated with a dried lipid mixture and then individual spots of DNA would merely have to be spotted onto the slide where they could precipitate in place.

Exemplary variations of the foregoing procedures are as follows:

| | Day | | |
|---|---|---|---|
| % Cell Transduction | 1 | 2 | 3 |
| Standard | 15% | 45% | 60% |
| 200 ul transfection mix | 15% | 45% | 65% |
| 100 ul transfection mix | 10% | 25% | 50% |
| 20 ul transfection mix | 3% | 15% | 25% |
| 2 × 10(5) cells | 15% | 40% | 50% |
| 0.2 × 10(5) cells | 15% | 25% | 25% |
| Optimem | 5% | 20% | 20% |
| 2% DMEM | 15% | 40% | 40% |
| Washed 2x with PBS | 15% | 45% | 45% |
| Lipofectamine Plus | 15% | 45% | 55% |
| Lipofectamine | 10% | 35% | 50% |
| 5 ul spot size | 10% | 30% | 40% |
| 1 ul spot size | 3% | 10% | 20% |
| 0.25 ul spot size | 3% | 25% | 20% |
| 0.1 ul spot size | 1% | 5% | 5% |

What is claimed is:

1. An array of viable cells on a substrate;
a. each element of said array comprising a subset of the cells transduced with a preselected nucleic acid;
b. the identity of each of said transduced nucleic acids being known in relation to the location of the element on the substrate;

2. The array of claim 1 wherein the preselected nucleic acids comprise a cDNA library, a viral vector library, a retrovirus library, an adenovirus library, an RNA library, an oligonucleotide library, a library from a virus, an agrobacterium library, or an antisense library.

3. The array of claim 1 wherein the preselected nucleic acids comprise the identical gene mutated at defined genetic locations at each element of the array.

4. The array of claim 1 wherein the identities of at least some of the preselected nucleic acids are known.

5. The array of claim 1 wherein the preselected nucleic acids have been frozen on the substrate.

6. The array of claim 1 wherein the preselected nucleic acids are selected for their likelihood of inhibiting an identified disease state, phenotype or condition.

7. The array of claim 1 wherein the preselected nucleic acids are selected for their being associated with a known or suspected biological function.

8. The array of claim 1 wherein the preselected nucleic acids encode membrane proteins.

9. The array of claim 1 wherein the preselected nucleic acids encode G-protein couple receptors, ion channels, or viral Envelope proteins.

10. The array of claim 1 wherein the cells are mammalian, avian, insect, plant, plant protoplast, yeast, fungus, bacterium, or human.

11. The array of claim 1 wherein the cells are made to express a gene of known identity prior to their application to the substrate.

12. An array of nucleic acids on a substrate, the identity of the elements of the array being known in relation to the location of the elements on the substrate; each element of the array further comprising a gene transduction vehicle.

13. The array of claim 12 wherein the nucleic acids are DNA.

14. The array of claim 12 wherein the nucleic acids comprise a cDNA library, a viral vector library, a retrovirus library, an adenovirus library, an RNA library, an oligonucleotide library, a library from a virus, an agrobacterium library, or an antisense library.

15. The array of claim 12 wherein the nucleic acids comprise the identical gene mutated at defined genetic locations at each element of the array.

16. The array of claim 12 wherein the identities of at least some of the nucleic acids are known.

17. The array of claim 12 substantially stable to freezing conditions.

18. The array of claim 12 wherein the substrate is compatible with use for mass spectrometry.

19. The array of claim 12 wherein the substrate further comprises a gold layer.

20. A solid body having a surface, said surface being adapted;
a. to bind a gene transduction vehicle in a reversible manner;
b. to permit cells to adhere to the surface; and
c. to allow said cells to be transduced by said tansduction vehicle;

21. The solid body of claim 20 wherein the adaptation comprises an antibody directed to the transduction vehicle.

22. The solid body of claim 20 wherein the adaptation comprises an antibody directed to the exterior proteins of a viral vector.

23. The solid body of claim 20 wherein the adaptation comprises an antibody directed to the Hexon or Fiber proteins of Adenovirus.

24. A solid body having a surface treated to allow spotting of volumes of liquid less than about 1 microliter in an array format without allowing the liquid to completely desiccate.

25. The solid body of claim 24 wherein the treatment comprises application of trehalose, gama-amino-propylsilane, or freezing.

26. A method for spotting volumes of liquid less than about 1 microliter in an array format onto a solid surface without allowing the liquid to completely desiccate, comprising including in the spotting medium trehalose or glycerol.

27. A method of constructing an array of viable cells comprising:
a. providing a substrate;
b. elaborating upon the substrate an array of nucleic acids;
c. binding viable cells to the substrate at the locations where elements of the array of nucleic acids are present; and
d. transducing at least some of the cells present at said locations with the nucleic acid present at those locations.

28. The method of claim 27 further comprising including a gene transduction enhancing composition with the nucleic acids elaborated upon the substrate.

29. The method of claim 27 further comprising coating a surface of the substrate with a binding promoting composition to enhance the binding of the array of nucleic acids to the substrate.

30. The method of claim 27 further comprising incubating the array subsequent to transduction under conditions selected to promote growth of the cells.

31. The method of claim 27 wherein the cells are mammalian, avian, insect, plant, plant protoplast, yeast, fungus, bacterium, or human.

32. The method of claim 27 wherein the substrate is inorganic.

33. The method of claim 27 wherein the substrate is glass.

34. A method for determining the biological products produced by members of a library of nucleic acids comprising:
a. constructing an array of viable cells comprising:
b. elaborating upon a substrate an array comprising at least a portion of said library of nucleic acids;
c. binding viable cells to the substrate at the locations where elements of the array of nucleic acids are present;
d. transducing at least some of the cells present at said locations with the nucleic acid present at those locations;
e. incubating the array of cells under conditions selected to promote growth of the cells;
f. determining the biological products produced at elements of the array; and
g. relating the production of such elements with the nucleic acid present at said elements.

35. The method of claim 34 wherein the identities of the members of the library of nucleic acids are known in relation to the location of the nucleic acids on the substrate.

36. The method of claim 34 wherein the library of nucleic acids is selected to be related to a disease state

37. The method of claim 34 wherein the library of nucleic acids is selected to be associated with a known or suspected biological function

38. The method of claim 34 wherein the library of nucleic acids is used to identify a protein mutation with a defined phenotype or function.

39. The method of claim 34 wherein the library of nucleic acids is selected to encode surface-bound monoclonal antibodies.

40. The method of claim 34 wherein the array is used to identify drug candidates that bind to proteins correlated with adverse absorption, digestion, metabolism, excretion, toxicity, bioavailability, or cell death.

41. The method of claim 34 wherein the library of nucleic acids comprises the identical gene mutated at defined genetic locations at each element of the array.

42. The method of claim 34 wherein said elaboration includes placing upon a surface of the substrate a binding promoting composition to enhance the binding of the nucleic acids to the substrate.

43. The method of claim 34 wherein said elaboration includes co-depositing a gene transduction enhancing composition with the nucleic acids on the substrate.

44. The method of claim 34 wherein said relating comprises detecting the biological products produced by the cells.

45. The method of claim 34 wherein the array is challenged with a predetermined chemical or biological species during at least part of the incubation step.

46. The method of claim 34 performed under the operative control of a computer.

47. The method of claim 34 performed to identify a protein mutation with a select phenotype.

48. The method of claim 47 wherein the phenotype is improved antibody reactivity for use in designing improved vaccine candidates.

49. The method of claim 34 wherein the array is used to identify the target for a drug candidate where said target is not yet linked to a specific disease.

50. The method of claim 34 wherein the array is used to identify the target for a drug candidate of unknown specificity.

51. The method of claim 50 wherein the drug candidate is a protein, monoclonal antibody, or low-molecular weight organic compound.

52. The method of claim 50 wherein the drug candidate has been tested for toxicity and bioavailability prior to the identification of its target.

53. The method of claim 34 wherein the cells stably express a gene of known identity prior to their application to the substrate.

54. The method of claim 34 wherein the cells contain a co-transduced gene, comprising one or more genes introduced into all the cells used on the array.

55. The method of claim 54 wherein the co-transduced gene is a modifying enzyme.

56. The method of claim 54 wherein the co-transduced gene is a kinase, phosphatase, glycosidase, protease, or chaperone protein.

57. The method of claim 34 wherein the array is used to define antibody reactivities from an animal's sera.

58. The method of claim 34 wherein the nucleic acid library is derived from one species and the cells are derived from a different species.
